(19) United States
(12) Patent Application Publication
(10) Pub. No.: US 2024/0343929 A1
(43) Pub. Date: Oct. 17, 2024

(54) MATRIX

(71) Applicant: OPES CORPORATION OY, ESPOO (FI)

(72) Inventors: Janne JARVENPAA, Veikkola (FI); Kari HOLOPAINEN, Lahti (FI)

(21) Appl. No.: 18/755,021

(22) Filed: Jun. 26, 2024

Related U.S. Application Data
(62) Division of application No. 14/898,396, filed on Dec. 14, 2015.

(30) Foreign Application Priority Data
Jun. 13, 2013 (WO) PCT/FI2013/050649

(51) Int. Cl. C09D 11/14; A61K 8/02; A61K 8/73; A61Q 13/00; B41M 3/00; C09D 5/00; C09D 11/03; C09D 11/30

(52) U.S. Cl. CPC C09D 11/14; A61K 8/0216; A61K 8/731; A61Q 13/00; B41M 3/006; C09D 5/00; C09D 11/03; C09D 11/30

(57) ABSTRACT

A matrix for dissolving including a functional compound, method of preparing and using such a matrix, and a method of preparing a product including a functional compound as well as the use of such a product are described. The matrix has been provided with at least one functional compound, and at least one solvent dissolving the functional compound, and at least one film-forming agent, wherein the solvent dissolving the functional compound also homogenizes the film-forming agent into the matrix.

### CROSS REFERENCE TO RELATED APPLICATIONS

This application is a divisional of (and claims the benefit of) pending U.S. patent application Ser. No. 14/898,396, filed Dec. 14, 2015, the disclosure of which is hereby incorporated herein by reference in its entirety. This application also claims the benefit of International Patent Application No. PCT/FI2014/050458, filed Jun. 6, 2014, which claims priority to Finnish Patent Application No. PCT/FI2013/050649, filed Jun. 13, 2013, both of which are incorporated herein by reference in their entirety.

### TECHNICAL BACKGROUND

The invention relates to a matrix comprising a functional compound as well as to a method of preparing and using of such a matrix. The invention also relates to a method of preparing a product comprising a functional compound and to the use of such a product.

Scents have traditionally been added to products by directly dosing them into the base material, or, by spraying scented compounds into a printed product, for example. The known technologies suffer from the problem that the dosing of the scented compound either is laborious and expensive or the scented compound cannot be dosed into the printed product in a sufficient amount. The scent can also be created by enclosing an odorizing agent in a capsule and by subsequently rubbing or abrading it. This method is also laborious and technically complicated. Besides, a method is known in which a scented compound is added to a product, included in printing ink, varnish and/or glue. In the methods, it is necessary to either activate the product or to install/remove a separate protective film in order to enable a controlled release of the scent.

### GENERAL DESCRIPTION OF THE INVENTION

Now, a matrix comprising a functional compound has been invented, which matrix is technically especially advantageous for dosing and releasing the functional compound. The invention also relates to a method of preparing, and to the use of, such a matrix, as well as to a method of preparing a product comprising a functional compound, and to the use of such a product. In order to achieve this objective, the invention is characterized by what is set forth in the independent claims. The rest of the claims disclose some preferable embodiments of the invention.

According to an object of the invention, matrix has been provided with at least one functional compound, and at least one solvent dissolving the functional compound, and at least one film-forming agent, wherein said solvent dissolving the functional compound also homogenizing the film-forming agent into the matrix.

The matrix is a technically especially advantageous way of dosing and releasing a functional compound. The matrix according to the invention makes it possible to dissolve, as well as to dose, functional compounds, such as scents, and to release them from different substrates and products in a film-assisted, controlled way. Functional compound is dissolved to the solvent so that it is homogenous and formed solution thus comprises all the properties of the functional compound, including e.g. first note, heart note and base note. This gives essential advantage compared to known art. To said solution the film forming agent is added to make it to be homogenized matrix, the use of which is especially advantageous for dosing and releasing functional compound. Such a matrix is mixable with a very large variety of compounds. The release of the functional compound can be controlled and adjusted by changing the thickness and properties of the film formed by means of the matrix. According to an object of the invention, the nature of the odor can also be fine adjusted by changing the thickness and properties of the film. The matrix/product is usable for a desired period of time, without any separate activation. Thus, it has very wide and substantial applications and possibilities of implementation.

The matrix is usable, as such, for dosing a functional compound, or, it is usable for manufacturing a product by printing or by dosing in some other way. A permeable film can preferably be provided on the drying matrix and/or product, in order to lower the diffusion rate of the functional compound and in order to release it during an extended period of time compared to a filmless product. The matrix/product is preferably usable as a scented product, odor regulator/remover, repellent, attractant and/or pesticide, taste, or taste eliminator. The matrix alone is also usable for preventing or adjusting the release of functional compounds. Besides, the product can be provided with a plastic film or a paraffin film, for example, to further regulate the release of the functional compound.

According to an object of the invention amount of film-forming agent in matrix is below 5%, such as below 2%, as preferable below 1%. Then its properties are suitable for dosing functional compound or it can be used for manufacturing a product by printing or by dosing in some other way.

According to an object of the invention, the film-forming agent is ethyl cellulose. It forms a stable permanent film that is clear, UV stable, i.e. non-yellowing, highly temperature-resistant from −70° C. to 150° C. (screenprinting 60° C. and textile printing 140° C.), inexpensive and safe to use. Ethyl cellulose in itself does not dissolve in water but can be get there by means of a solvent, such as ethanol.

According to an object of the invention, the solvent is an alcohol. According to an object of the invention, the solvent is ethanol. Preferably, the ethanol is as waterfree as possible or completely waterfree. Alcohols are good solvents for a large variety of compounds. Ethanol is safe to use because it gives a non-toxic matrix. It in itself is almost odor free and technically easy to vaporize. The choice of solvent/solvents and film-forming agents depends on the functional compound desired. Some solvents/film-forming agents are usable for a very large variety of compounds. A preferred combination is ethanol as the solvent and ethyl cellulose as the film-forming agent.

According to an object of the invention, at least one scent is provided as a functional compound in the matrix. The scent may vary to a large extent. The choice of solvent/solvents and film-forming agents depends on the scent desired. Some solvents/film-forming agents are usable for a very large variety of compounds. According to an object of the invention, the scent is allergy-tested and/or such that it does not cause allergy, oversensitivity, sensitivity to light or general irritation to those subjected to it. Such products are safe to use in many different applications. Preferably, the scent can also be such that is suitable for use in foodstuffs.

According to an object of the invention, at least one deodorizing agent, repellent, attractant or pesticide, taste or taste regulator is provided as a functional compound in the matrix. Such applications can be used in product treatment, research or fishing/hunting, or, for killing or attracting insects or rodents, for instance. It is also possible to combine an attractant and a pesticide in the same product. Still, it is possible to manufacture a product that simultaneously repels some animals and attracts other animals.

According to an object of the invention, the matrix comprises at least one viscosity regulator. This widens the use of the invention because the matrix thereby also becomes usable where the matrix and the functional compound to be added need to have a certain viscosity in order to be dosable. Viscosity can be regulated individually by many ways/compounds. According to an object of the invention, the viscosity regulator is a silicate compound. The silicate compound changes the viscosity of the product and may further enhance the binding of the scented compound to the solution. Different silicate compounds can be used for different purposes.

According to an object of the invention, the matrix present in the product comprising a functional compound is printed, or dosed in some other way, into the product by a method chosen from a group including offset technology, screenprinting technology, flexo printing technology, inkjet/digital printing technology, tampo printing technology, gravure printing, relief printing, rotary printing, spray preparation, mist preparation, dipping.

According to an object of the invention, at least one printing agent is provided in the matrix. Matrix can be added to different printing technology as such e.g. to water-based or solvent-based varnish. The product can vary to a very large extent. It can be, for example, a product that is in use in the initial processing of a product, or in the final processing of an already finished product, or, it can be in use in mixed printing technology. The compound can be dosed in an especially accurate manner and the operation of the process can be adjusted, both in terms of the scented compound itself, and, of all the rest, according to any current requirements. The printing into the product is a very easy and technically simple solution. No separate equipment is needed for the printing, but still, the same very great advantages related to the printing technology are achieved as in printing in general. Therefore, the addition of the compound/compound is technically highly efficient. At the same, it is very reasonable from the economic point of view. For example, the method can produce shirts which then are printed with a desired design or color, for instance. Thus, this novel method gives, also in this respect, a substantial advantage compared to the known art.

According to an object of the invention, the manufactured product comprising a scented compound can be used, for example, as a product belonging to a group including fibrous products, such as textile, fabric, felt, cotton, accessory, sweatband, wristband, piece of cloth, trousers, shirt, cap, beanie, headgear, holdall, bag, cardboard beer mat, cardboard picture, vending rack, storage box, display box, brochure and catalogue, album, folder, mobile, table rack, floor rack, price wobbler, product box, packaging material, casing, pack, leaflet, calendar, ruler, display, container, dispenser, coaster, business card, form, envelope, catalogue, desk mat, plastic folder, briefcase, pouch, background advertisement, product information card, tablecloth, table talker, box or any other means or product designed for sales promotion, marketing, direct mail. The product can be a semi-finished product or a finished product and intended for sales and marketing, for example, or even for consumers.

According to an object of the invention, the at least one printing agent is a varnish from a group including solvent varnish, protective varnish, colorless varnish, translucent varnish, opaque varnish, colored varnish, printing varnish, paper varnish, textile varnish, offset varnish, dispersion varnish, UV varnish. It is especially preferable to use varnish as the printing agent because it allows, both technically and economically, the functional compound/matrix to be added to a large variety of products. According to an object of the invention, the at least one printing agent is printing ink or glue. This allows even more ways of adding the functional compound/matrix. Besides, it also becomes possible to influence on the intensity and duration of the release of the functional compound. According to an object of the invention, the at least one printing agent is a solvent-based, water-based and/or resin-based printing agent used in digital printing. This is an application of great importance because it allows functional compounds, such as scents, to be added in an important field of the printing technology.

According to an object of the invention, at least one carrier, such as spray gas carrier or mist carrier, is provided in the matrix. The carrier allows the functional compound/matrix to be dosed into textiles or cardboard without any printing technology. Besides, it makes it easy to repeat any desired treatment at regular intervals, such as every 1 or 2 months.

According to an object of the invention, the solvent is vaporized out of the matrix/product. The vaporization can be carried out either passively or actively. Then, a film is formed on the matrix, which, preferably without any separate activation, controls the release of the functional compound. According to an object of the invention, the product is ovened after the printing. This speeds up the vaporization/drying and the manufacture in general. It also enhances the manageability of the products.

According to an object of the invention, the matrix also has been provided with a surfactant, as a binding agent, for mixing and binding the carrier. The surfactant is preferably an anionic surfactant. The surfactant is preferably SDS (sodium lauryl sulphate). The SDS content can be, for example, 0.5 to 2% (w/v), preferably over 0.8% (w/v) or over 1% (w/v), for instance.

According to an object of the invention, accelerators are added to the matrix to enhance the binding of the scented compound. The method is thereby ensured, adjusted and enhanced to be optimal.

The base material of the product can be chosen, for example, from a group including paper, paperboard, cardboard, wood product, plastic, rubber, metal, glass, ceramic product, textile. According to an object of the invention, the product is a fibrous product, preferably a textile, for example. The method is especially suitable for printing textiles, for example. According to an object of the invention, at least one printing ink, glue and/varnish is also printed on the base material of the product by some other printing technology. Thus, the method is very useful for a large variety of applications.

Compared to the known art, the product manufactured by the method has the special advantage of being capable of easily and accurately controlling the amount of the compound in the product. In addition, the way that the printing is done results in the advantage of allowing control of the release thereof. As an example, it is possible to manufacture a product in which a scent has been printed, by screenprinting, into the printing inks and then partially coated by breathable varnish. The scent is released from the product over a long time span. The scent can also be printed into the product in a combination with the surface varnish to make it release relatively faster and in higher concentrations.

The scent can be any substance, compound or mixture causing at least some odor that can be sensed or measured. It can comprise only one such substance, mixture or compound or it can comprise two or more substances, compounds and/or mixtures in different proportions. The scent can also be a plant or a part thereof comprising one or more odor-causing substances, compounds and/or mixtures. The scent can also be a plant extract, for example. In this patent application, a scent refers to a compound which, mainly or at least partly, causes this scent. However, such a scent can vary, for example, an apple scent can be the scent of an acidic apple or the scent of a sweet apple, and bread, for instance, can have a wide range of scents.

According to an object of the invention, the product printed by a printing technology or the product dosed with a compound in some other way, is also provided with raised characters, recesses and perforations, such as embossed characters by braille technology or some other equivalent technology. Preferably, this provides combinations for the blind and the visually impaired, for example. Preferably, it is also useful where the product is handled in the dark, for example.

According to an object of the invention, the scent comprises one or more organic scented compounds. These allow a wider range of applications for the invention. As an example, they can create scent combinations which otherwise would have to be done at the scent raw material level. By combining the scent of vanilla with the scent of raw tobacco, for example, a combination resembling a vanilla cigar is obtained. According to an object of the invention, the scent comprises one or more scented inorganic compounds. These allow an even wider range of applications for the invention.

According to an object of the invention, the at least one ink is chosen from the following group: paper inks, plastic inks, textile inks, UV inks. The paper inks can be solvent and water based. Further, it possible to use inks with different gloss levels, such as inks giving a matte or glossy finish. Further, it is possible to use scratch-off ink, which is used, for example, on lottery tickets, or fluorescent ink, which absorbs light and glows in the dark, or light-reflective inks. This embodiment is useful, for example, where it is desirable to attract by both color and smell at the same time, such as in the manufacture of fishing lure coatings, or in luminous tags with scent, or in scented hunting vests intended to block the human scent which animals want to stay away from.

Basically, all plastic inks are solvent-based. The plastic inks can also be either matte or glossy. It is also possible to use, for example, two-component plastic inks on metal surfaces. Further, pigment or base paste inks, which are used for toning down colors and or as a base color, can be chosen from the plastic inks.

The textile inks are usually divided into plastisol, 1 or 2 component as well as watercolor-based inks. Further, it is possible to use different kinds of opaque inks, fluorescent inks and thinners.

The invention can also utilize UV drying inks. They are equivalent to the other inks but do not dry if not exposed to UV light.

The inks can be blended at the printing factory or the printing factory can order ink with a certain shade directly from an ink supplier.

According to an object of the invention, one or more scented compounds chosen from the following group are added:
the scent of a tar product, such as tar water or pit tar,
the scent of a smoke, such as chimneyless sauna or tar smoke,
the scent of a drink, such as coffee, cocoa, tea, mead, wine or beer,
the scent of a plant, such as mint or birch,
the scent of a spice, such as garlic, black pepper or white pepper,
the scent of a fruit, such as apple,
the scent of a flower, such as rose or lily-of-the-valley,
the scent of a foodstuff, such as bread, bun, smoke-cured meat or smoked fish,
the scent of an animal, such as horse,
the scent of a berry, such as raspberry.

The invention has very wide application possibilities for most different purposes. These make the invention even more useful.

### DETAILED DESCRIPTION OF THE INVENTION

### Formulation of Scent Compounds into Paper and Textile Varnish

The chemicals used were as follows:
P (varnish): Aquacolor Gloss WB381 (Sericol)
Txt (varnish): Texopaque OP00A (Sericol)

### Emulgators:

Sodium dodecyl sulphate SDS (Sigma)
Montanox 20 and Montanox 80 (Seppic Food grade)
Amidet N (Kao)

### Rheology Modifiers:

Aerosil 200 (Evonik)
Aerosil COK 84 (Evonik)
Sylothix 51 (JRS)

### Scents:

Parfum Coffee Latte (Holland Aromatics)
Parfum Wild Strawberry (Holland Aromatics)
Parfum Apple (Holland Aromatics)

### Film-Forming Agents:

Aqualon EC N10 (Hercules, pharm. grade)
Natrosol 250 HR (Ashland, pharm. grade)
Benecel A4C (Ashland, pharm grade)

### Measuring Equipment

The measuring instruments, water baths and buffer solutions were calibrated and under quality control.
Brookfield Model DV-II+ Viscometer (dynamic)
temperature-controlled water bath Julabo
pH-meter Phenomenal VWR
buffer solutions with pH 4, 7 and 11 AVS Titrinorm BDH Prolabo
GC double column mass spectrometry headspace technology
Agilent 5973 N MS-GC
Purge & Trap Consentrator

### Recipe (KA Aroma):

P—70 wt % P (paper varnish)
30 wt % of a mixture as follows:
90 wt % scent
10 wt % SDS (20 wt % water solution)
T—70 wt % T (textile)
30 wt % scent

### Initial Testing Results:

the scents were poorly dissolved in water and varnish P,
the scents were directly dissolved in varnish in a test that used varnishes from the product family of Sericol, intended for both paper and textile,
P comprised solvents of which 10 wt % vaporizable of which 10 wt % flammable 1-metoxy-2-propanol and water
T comprised slowly vaporizing solvents, of which 20 to 25 wt % Xi-classified bis (2-ethylhexyl) maleate, and a small amount of complexed Zn tall oil fatty acid aluminum chloride derivate as a catalyst.
it was observed that different scents required different amounts of emulgators in order to emulgate into varnish P,
it was observed that the Tr lauryl version was not stable but the viscosity varied from 100 000 to 200 000 MPas (25° C.)

For example, for the scent Latte the color of which also allowed the stability to be observed,

### Pr:

700 g P
250 g Latte
50 g (20 wt % SDS water solution)

### Tr:

the preceding solution with added 2 wt % Aerosil 200
   Viscosities (mPas, 25° C.):
P initial 12 600
Pr mixture final 11 800
T initial 16 800
Tr mixture 19 200
Tr mixture final 100 000 to 200 000

### pH Values:

scents 6 to 7 mildly acidic, drift
SDS 9.4
P and T 7
Pr 7
Tr mixture 5
Latte+SDS 3.3 to 3.4 SDS to the bottom, jellylike, reacted after a few days,
Strawberry+SDS 4.4
Apple+SDS 4.9

### Emulgators

NDS is an anionic emulgator. It, and the protons released from the scents, increase the salt concentration of the solution, with an impact on the solubility equilibrium. NDS was replaced with non-ionic emulgators. Polysorbate 20 (Montanox 20) and Polysorbate 80 (Montanox 80), which is even more oil-soluble, they both being easy-to-use when in liquid form, were tested. Amidet N (PEG-2 rapeseed oil amide derivative) was also tested. All of the emulgators resulted in unstable scent-dependent emulsions.

### Film-Forming Agents

Hydrocolloids that were non-ionic and had other appropriate properties were chosen as the film-forming agents (protonation of the scents). HEC (Natrosol 250 HR) which dissolves in water and HPMC (Benecol A4C) which dissolves in water and partially in solvents. There are commonly used as matrix release agents in medicinal chemistry.
Natrosol 250 HR (1 w % water solution) pH value 7.5 viscosity 2 900 mPas (25° C.)
Benecel A4C (1 w % water solution) pH value 7.4 viscosity 60 mPas (25° C.)

### Rheology Modifiers

As rheology modifiers, Aerosil 200 (fumed silica), Aerosil COK 84 (a melt mixture of quartz and aluminum), showing higher resistance in a polar environment, Sylothix 52 (consists of polyethylene fibers).

Mixtures Pr and Tr were made with a film-forming agent
Pr+1 wt % Natrosol 250 HR viscosity 217 000 mPas and PH value 7
Pr+1 wt % Benecel A4C viscosity 120 000 mPas and pH value 7
Tr+1 wt % Benecel A4C+2% Aerosil 200 viscosity 260 000 mPas and pH value 5
Tr+1 wt % Natrosol 250 HR viscosity 14 000 mPas and pH value 5

### Results

the degree of protonation of the scents in water varies
when NDS as anionic emulgator, a stability problem with different scents
non-ionic emulgators Natrosol 250 HR and Benecel A4C
Natrosol 250 HR and Benecel A4C dissolve in Tr comprising SDS water solution and the Aerosil 200 amount required becomes smaller by half
Pr solutions with Natrosol 250 HR and Benecel A4C have excessive viscosity
scents cannot be emulgated into a stable mixture with varnishes and added into them

Mixtures Ti were made without an emulgator (70/30 =varnish T/scent):
210 g T
90 g Latte

Initial viscosity of the mixture 15 000 mPas
Ti+2 wt % Aerosil (6 g)->final viscosity 150 000 mPas
Ti+3 wt % Aerosil (9 g)->final viscosity 180 000 mPas
Ti+1 wt % Natrosol 250 HR viscosity 18 000 mPas
Ti+1 wt % Natrosol 250 HR+3 wt % Aerosil 200 viscosity 120 000 to 170 000 mPas (sails)
Ti+1 wt % Benecel A4C viscosity 17 000 mPas
Ti+1 wt % Benecel A4C+3 wt % Aerosil 200 viscosity 170 000 mPas

### Results

Benecel A4C can be used as a film-forming agent without any water emulgator for textile varnish

### Utilization of Extraction

A solvent perfectly dissolving scents in both aqueous and solvent phases was needed.
the solvent must be generally available, inexpensive and safe (with known toxicology)
the solvent must be “odorless”
the solvent must vaporize from the matrix
the solvent must keep the film-forming agent homogenous in the solution
a permeable film structure must be formed while the solvent vaporizes
the agent on which the film is formed must be soluble in the film-forming agent
the film-forming agent must be generally available, inexpensive and safe (with known toxicology)

Both a straight-chain and a branched-chain alcohol were tested.

Homogenous solution Pf was made
68.3 wt % P
22.9 wt % scent
8.2 wt % ethanol (Industol)
0.61 wt % N10PH
A12, a purer grade of alcohol, can also be used (more odorless; with a different bitter element). A branched alcohol (such as 2-propanol) works as well but it has a more pungent smell and is needed in a triple amount.
Ethyl cellulose (N10.PH) does not dissolve in water but can be get there by means of an alcohol. It forms a permanent film which is clear, UV stable, i.e. non-yellowing, temperature-resistant from −70° C. to 150° C. (screenprinting 60°° C. and textile printing 140° C.), inexpensive and safe.

### Tf:

Homogenous solution Tf was made in the same way as Pf
68.3 wt % T
22.9 wt % scent
8.2 wt % ethanol (Industol)
0.61 wt % N10PH

If needed, the ethanol content can be decreased down to 5.9 wt % (the varnish comprises solvents).

Solution Tf was made thicker
Aerosil 200 viscosity does not increase; e.g 2 wt % 53 000 mPas (25° C.)
Sylothix 51 4 wt % 40 000 mPas (25° C., becomes granular)
   do not work in a polar environment
   Aerosil COK 84 silicon aluminum oxide for a polar environment
Tf thickened 3.7 wt % 130 000 mPas (25° C.)

Note also that the scent can be perfectly dissolved in P with a small amount of alcohol. This allows the scent to be concentrated into varnish:
68.3 wt % P
29.3 wt % scent (Latte)
2.4 wt % ethanol

The scent (Latte) could be concentrated into textile varnish in an amount of 40%.
60.0 wt % T
40.0 wt % scent (Latte)

For paper varnish and textile varnish, the following mixture can be used:
22.9 wt % scent
8.2 wt % ethanol (Industol)
0.61 wt % N10PH

For paper varnish
68.3 wt % P

A ready mixture as follows
72.3 wt % scent
25.8 wt % ethanol
1.92 wt % N10PH is mixed with the paper varnish in an amount of 31.7 wt %.

For textile varnish
68.3 wt % T and thickening with 3.7 wt % Aerosil COK 84

A ready mixture as follows
72.3 wt % scent
25.8 wt % ethanol
1.92 wt % N10PH is mixed with the textile varnish in an amount of 31.1 wt %, then thickening with 3.7 wt % Aerosil COK84.

In the paper and textile varnishes, the content of N10PH, once the alcohol has vaporized, is 0.82 wt % (initially 0.61 wt %).

### Printing and Analysis

Solutions were made for printing and analysis (the weights are in grams).

| | P1 | P2 | P3: |
|---|---|---|---|
| P (varnish) | 102.45 | 102.45 | 102.45 |
| Latte | — | 34.35 | 34.35 |
| Industol | 12.40 | 12.30 | 12.30 |
| N10PH | 0.92 | — | 0.92 |

These were printed by screenprinting (screen: 60 yarns/cm², 50° C.) 3.4.2013 on (the coated side of) paperboard. The printing order was P1, P2 ja P3. After been taken out of the oven, the items in size A4 were dry and were cut into 1 cm×4 cm strips. The strips were inserted into airtight vials. The scent of Latte and P1 were the standard and the matrix, respectively. 0.1 g more Industol had to be used in the matrix than in samples P2 and P3 to obtain a homogenous mixture. Two parallel measurements were made. The samples were analyzed for the release of the scent by head space GC-MS technology over a period of 0 to 4 weeks. The sample was heated at 30° C. for 10 minutes and the components were collected on a Tenax trap with a flow of helium. The components were desorbed from it into a cold trap (liquid nitrogen, −120° C.). The samples were stored in an odorless, well-ventilated place at room temperature.

The significant scent agents were 3-carene (fused six-membered and three-membered ring) ja d-limonene (six-membered ring), indane (fused aromatic six-membered and five-membered ring), straight-chain aldehyde octanal and solvent 1-meroxy-2-propanol.

### Results and Conclusions

The chosen film-forming technology works. The recipes are homogenous and useful for both paperboard and paper varnish applications. As the transfer of molecules through the film depends on their structure, it is impossible to determine how the scent precisely vaporizes through the film. Compared to the filmless samples, the vaporization was clearly reduced with the film. In the future, the marker can be any known scent compound, having an easy-to-determine retention time (the location of the peak) and content (the area of the peak). The risk of contamination should be avoided because citrus compounds, for example, are common in different detergents, such as hand and universal detergents. Longer research times impose a challenge in terms of both equipment adjustment and contamination.

The amount of D-limonene in a sample without film had dropped down to 0.51% in 2 weeks, while its content in a sample with film was 20.8%. The film had thus relatively lowered the diffusion rate by 86% (week 0) and by 79% (week 2). Note that each substance also has a characteristic scent threshold. For D-limonene it is 200 ppb.

The diffusion rate of 3-carene lowered by 51% (week 0)

The diffusion rate of indane lowered by 32% (week 0)

The diffusion rate of octanal was lowered by 36% (week 0)

The diffusion rate of the solvent lowered by 43% (week 0 and week 2)

The diffusion rate can be further lowered by increasing the thickness of the film. The research gives a good basis for optimizing the amount of the film-forming agent by means of a marker.

A product/matrix that is
standardized
applicable for different scents
applicable for different printing technologies
salable in a commercial and product-safe way
   is obtained.

### Combination of Offset Varnish and Scent

The applicability of the results from the scent project on paper and textile varnishes (27.5.2013) to offset technology was studied.

The chemicals used were as follows:
Offset (varnish O) Novacoat 4060 (FlintGroup)
Scents Parfum Apple (Holland Aromatics)
Film-forming agent Aqualon EC N10 (Hercules, pharm. grade)

### Measuring Equipment

The measuring instruments, water baths and buffer solutions were calibrated and under quality control.
Brookfield Model DV11+Viscometer
temperature-controlled water bath Julabo
pH-meter Phenomenal VWR
buffer solutions with pH 4, 7 and 11 AVS Titrinorm BDH Prolabo

### General

O is an oil-based varnish with 10 to 20% light paraffinic solvents
the varnish also comprises, as a catalyst, cobalt-2-ethylhexacyanate
O dries through an oxidation reaction
the kinematic viscosity of O is 7 cSt (40° C.)

### Results

A scent could be concentrated into the offset varnish in an amount of 56 wt % without alcohol. The textile and offset varnishes comprise solvents allowing the scent to be directly dissolved in the varnish.

Dynamic viscosities (mPas, 25° C.) and pH value
O: 56 000 mPas/pH value 6.0
mixture: 25 500 mPas/pH value 6.2
68.3 wt %
22.9 wt % scent
8.2 wt % ethanol
0.61 wt % N10PH

A mixture was made with
22.9 wt % scent
8.2 wt % ethanol
0.61 wt % N10PH

This was mixed with the offset varnish
68.3 wt %

That is, a ready mixture with
72.3 wt % scent
25.8 wt % ethanol
1.92 wt % N10PH
   was mixed with the offset varnish 31.7 wt %

The content of N10PH in the offset varnish, after the vaporization of the alcohol, is 0.82 wt % (initially 0.61 wt %). The mixture is fully homogenous. The paper, textile and offset varnish matrices are uniformly applicable.

1. A functional compound comprising fibrous product, the fibrous product comprising:
a fibrous substrate; and
a matrix comprising:
at least one functional compound, wherein the at least one functional compound comprises at least one scent;
an ethyl cellulose film-forming agent, wherein the amount of film-forming agent in the matrix is below 2%; and
a viscosity regulator;
wherein the matrix is printed on the fibrous substrate and forms a permeable film that lowers the diffusion rate of the functional compound from the fibrous substrate.

2. The functional compound comprising fibrous product of claim 1, wherein a solvent has been vaporized out of the matrix in the product.

3. The functional compound comprising fibrous product of claim 1, wherein the viscosity regulator is a silicate compound.

4. The functional compound comprising fibrous product of claim 1, wherein the amount of film-forming agent in the matrix is below 1%.

5. The functional compound comprising fibrous product of claim 1, wherein the matrix is printed on the fibrous substrate by a method selected from the group consisting of screenprinting, flexo printing technology, inkjet/digital printing technology, tampo printing technology, gravure printing, relief printing, and rotary printing.

6. The functional compound comprising fibrous product of claim 1, further comprising at least one printing agent selected from the group consisting of:
a varnish selected from the group consisting of: solvent varnish, protective varnish, colorless varnish, translucent varnish, opaque varnish, colored varnish, printing varnish, paper varnish, textile varnish, offset varnish, dispersion varnish, and UV varnish;
a printing ink or glue; and
a printing ink for digital printing selected from the group consisting of a solvent-based printing ink, a water-based and a resin-based printing ink.

7. A method of preparing a functional compound comprising fibrous product, the method comprising:
providing a fibrous substrate;
providing a matrix comprising:
at least one functional compound, wherein the at least one functional compound comprises at least one scent,
an ethyl cellulose film-forming agent, wherein the amount of film-forming agent in the matrix is below 2%,
a viscosity regulator, and
a solvent; wherein the solvent dissolves and homogenizes the at least one functional compound and the film-forming agent into the matrix;
printing the matrix on the fibrous substrate;
vaporizing the solvent from the printed matrix; and
forming a permeable film on the fibrous substrate that lowers the diffusion rate of the functional compound from the fibrous substrate.

8. The method of preparing a functional compound comprising fibrous product of claim 7, wherein the viscosity regulator is a silicate compound.

9. The method of preparing a functional compound comprising fibrous product of claim 7, wherein the amount of film-forming agent in the matrix is below 1%.

10. The method of preparing a functional compound comprising fibrous product of claim 7, wherein the matrix is printed on the fibrous substrate by a method selected from the group consisting of screenprinting, flexo printing technology, inkjet/digital printing technology, tampo printing technology, gravure printing, relief printing, and rotary printing.

11. A functional compound comprising fibrous product, the fibrous product comprising:
a fibrous substrate; and
a matrix comprising:
at least one functional compound, wherein the at least one functional compound comprises at least one scent;
an ethyl cellulose film-forming agent, wherein the amount of film-forming agent in the matrix is below 2%; and
a viscosity regulator;
wherein the matrix is dosed on the fibrous substrate and forms a permeable film that lowers the diffusion rate of the functional compound from the fibrous substrate.

12. The functional compound comprising fibrous product of claim 11, wherein a solvent has been vaporized out of the matrix in the product.

13. The functional compound comprising fibrous product of claim 11, wherein the viscosity regulator is a silicate compound.

14. The functional compound comprising fibrous product of claim 11, wherein the amount of film-forming agent in the matrix is below 1%.

15. The functional compound comprising fibrous product of claim 11, wherein the matrix is dosed on the fibrous substrate via printing.

16. The functional compound comprising fibrous product of claim 15, wherein the matrix is printed on the fibrous substrate by a method selected from the group consisting of screenprinting, flexo printing technology, inkjet/digital printing technology, tampo printing technology, gravure printing, relief printing, and rotary printing.

17. The functional compound comprising fibrous product of claim 11, wherein the matrix is dosed on the fibrous substrate by a method selected from the group consisting of offset technology, spray preparation, and mist preparation.

18. The functional compound comprising fibrous product of claim 11, further comprising at least one printing agent selected from the group consisting of:
a varnish selected from the group consisting of: solvent varnish, protective varnish, colorless varnish, translucent varnish, opaque varnish, colored varnish, printing varnish, paper varnish, textile varnish, offset varnish, dispersion varnish, and UV varnish;
a printing ink or glue; and
a printing ink for digital printing selected from the group consisting of a solvent-based printing ink, a water-based and a resin-based printing ink.
